(19) United States
(12) Patent Application Publication
(10) Pub. No.: US 2024/0074722 A1
(43) Pub. Date: Mar. 7, 2024

(54) METHOD FOR OBTAINING TUBE CURRENT VALUE AND MEDICAL IMAGING SYSTEM

(71) Applicant: GE Precision Healthcare LLC, Waukesha, WI (US)

(72) Inventors: Jingting Li, Beijing (CN); Xue li Wang, Beijing (CN); Bingjie Zhao, Beijing (CN)

(21) Appl. No.: 18/457,973

(22) Filed: Aug. 29, 2023

(30) Foreign Application Priority Data
Aug. 31, 2022 (CN) 202211053248.5

(51) Int. Cl. A61B 6/00; G06T 11/00; G16H 40/63

(52) U.S. Cl. CPC A61B 6/545; A61B 6/488; G06T 11/005; G16H 40/63

(57) ABSTRACT

The present application provides a method for obtaining a tube current value, a medical imaging system, and a non-transitory computer-readable storage medium. The example method for obtaining a tube current value includes obtaining a scanning protocol, performing a scout scan to obtain a scout image of a subject under examination, and obtaining a tube current value on the basis of a trained machine learning model, according to the scout image, the scanning protocol, and a preset image noise parameter.

### CROSS-REFERENCE TO RELATED APPLICATIONS

This application claims priority to Chinese Application No. 202211053248.5, filed on Aug. 31, 2022, the disclosure of which is incorporated herein by reference in its entirety.

### TECHNICAL FIELD

The present invention relates to medical imaging, and relates in particular to a method for obtaining a tube current value, a CT scanning method and a medical imaging system.

### BACKGROUND

In the process of computed tomography (CT), a detector is used to acquire data of X-rays passing through a subject under examination, and then the acquired X-ray data is processed to obtain projection data. The projection data may be used to reconstruct a CT image. Complete projection data can be used to reconstruct an accurate CT image for diagnosis.

In dose control of X-rays, automatic exposure control (AEC) is generally used to control the current of an X-ray source so as to control the dose of exposure. For different images under examination, it is generally necessary to use different scan parameters to perform exposure so as to obtain medical images thereof, and for different scanning sites of the same subject under examination, it is also generally necessary to use different scan parameters to perform exposure.

The subject under examination is equated to a standard phantom, and then the selected protocol is corrected. For example, the subject under examination can be equivalent to a standard human phantom (e.g., a standard size human body), or different sites can be equivalent to elliptical phantoms of different sizes, or a contour is obtained on the basis of a camera. However, the structure of the human body itself is complex, that is, information regarding height, body shape and weight, etc., is different for different people, and there are certain differences between different sites or organs; and even for the same site or organ, proportions of various tissues therein are different. This results in a certain gap between the actual human body itself and an equivalent phantom. Even if the subject under examination is equivalent to a standard phantom, there is a certain error in the correspondence between the equivalent phantom and the tube current since the correspondence is obtained by means of human phantom measurement in a laboratory. Therefore, there is a certain error when obtaining the tube current using an equivalent phantom, and there is thus a certain error between the obtained noise of medical images and the expected noise.

### SUMMARY

The present invention provides a method for obtaining a tube current value, a CT scanning method and a medical imaging system.

An exemplary embodiment of the present invention provides a method for obtaining a tube current value, the method comprising: obtaining a scanning protocol; performing a scout scan to obtain a scout image of a subject under examination; and obtaining a tube current value on the basis of a trained machine learning model, according to the scout image, the scanning protocol, and a preset image noise parameter.

An exemplary embodiment of the present invention further provides a CT scanning method, the method comprising: obtaining a scanning protocol; performing a scout scan to obtain a scout image of a subject under examination; obtaining a tube current value on the basis of a trained machine learning model, according to the scout image, the scanning protocol, and a preset image noise parameter, so as to obtain an updated scanning protocol; and performing a CT scan on the basis of the updated scanning protocol to obtain a medical image of the subject under examination.

An exemplary embodiment of the present invention also provides a medical imaging system, the system comprising a processor, and the processor executing the described method for obtaining a tube current value.

An exemplary embodiment of the present invention further provides a medical imaging system, the medical imaging system comprising: a scanning module, a user interface module and a control module; the scanning module is used to perform a scout scan to obtain a scout image, and to perform a main scan to obtain a medical image; the user interface module is configured to select a scanning protocol and to select a preset image noise parameter; and the control module is used to obtain a tube current value on the basis of a trained machine learning model, according to the scout image, the scanning protocol, and the image noise parameter.

Other features and aspects will become apparent from the following detailed description, drawings, and claims.

### BRIEF DESCRIPTION OF THE DRAWINGS

The present invention can be better understood by means of the description of the exemplary embodiments of the present invention in conjunction with the drawings, in which:

FIG. 1 is a schematic diagram of a CT system according to some embodiments of the present invention;

FIG. 2 is a schematic diagram of a CT scanning process according to some embodiments of the present invention;

FIG. 3 is a schematic diagram of a medical imaging system according to some embodiments of the present invention;

FIG. 4 is a schematic diagram of the training of a machine learning model in a control module shown in FIG. 3;

FIG. 5 is a schematic diagram of an application of a machine learning model in a control module shown in FIG. 3;

FIG. 6 is a flowchart of a method for obtaining a tube current value according to some embodiments of the present invention; and

FIG. 7 is a flowchart of a CT scanning method according to some embodiments of the present invention.

### DETAILED DESCRIPTION

Specific embodiments of the present invention will be described below. It should be noted that in the specific description of said embodiments, for the sake of brevity and conciseness, the present description cannot describe all of the features of the actual embodiments in detail. It should be understood that in the actual implementation process of any embodiment, just as in the process of any one engineering project or design project, a variety of specific decisions are often made to achieve specific goals of the developer and to meet system-related or business-related constraints, which may also vary from one embodiment to another. Furthermore, it should also be understood that although efforts made in such development processes may be complex and tedious, for a person of ordinary skill in the art related to the content disclosed in the present invention, some design, manufacture, or production changes made on the basis of the technical content disclosed in the present disclosure are only common technical means, and should not be construed as the content of the present disclosure being insufficient.

Unless defined otherwise, technical terms or scientific terms used in the claims and description should have the usual meanings that are understood by those of ordinary skill in the technical field to which the present invention belongs. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise,” and do not exclude other elements or objects. The terms “connect” or “link” and similar words are not limited to physical or mechanical connections, and are not limited to direct or indirect connections.

As used in the present invention, the term “subject under examination” may include any object being imaged.

It should be noted that from the perspective of a person of ordinary skill in the art or related art, such descriptions should not be construed as limiting the present invention to only a CT system. In fact, the method and apparatus for obtaining a tube current value described here may be reasonably applied to other imaging fields in medical or non-medical fields, such as X-ray systems, PET-CT systems, SPECT systems, or any combination thereof.

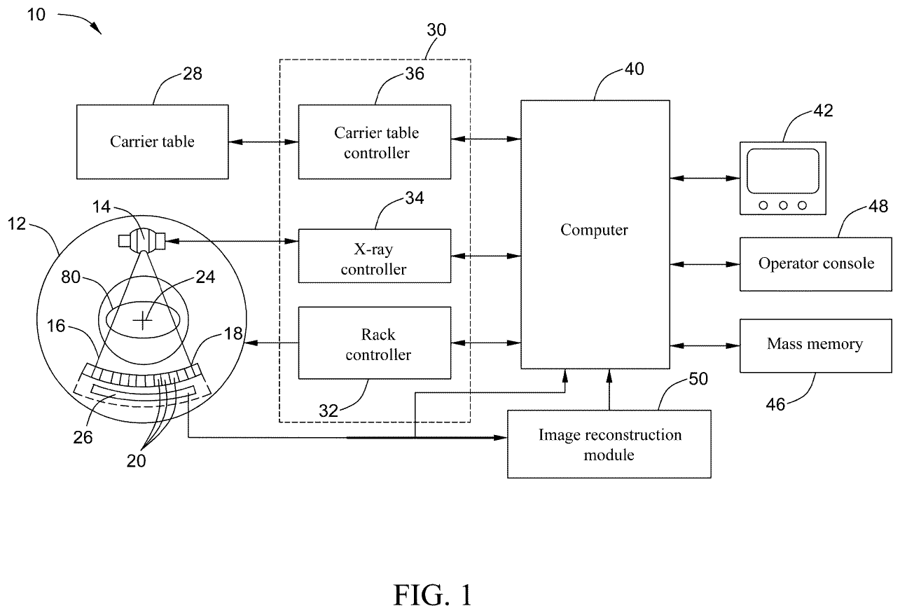

FIG. 1 shows a schematic diagram of a CT system 10 according to some embodiments of the present invention. As shown in FIG. 1, the system 10 includes a rack 12. An X-ray source 14 and a detector array 18 are disposed opposite to each other on the rack 12. The detector array 18 is composed of a plurality of detectors 20 and a data acquisition system (DAS) 26. The DAS 26 is used to convert sampled analog data of analog attenuation data received by the plurality of detectors 20 into digital signals for subsequent processing. In some embodiments, the system 10 is used for acquiring, from different angles, projection data of a subject under examination. Thus, components on the rack 12 are used for rotating around a rotation center 24 so as to acquire projection data. During rotation, the X-ray radiation source 14 is used to emit X-rays 16 that penetrate the subject under examination toward the detector array 18. Attenuated X-ray beam data is preprocessed and then used as projection data of a target volume of the subject. An image of the subject under examination may be reconstructed on the basis of the projection data. The reconstructed image may display internal features of the subject under examination. These features include, for example, a lesion, a size, and a shape of a body tissue structure. The rotation center 24 of the rack also defines the center of a scanning field 80.

The system 10 further includes an image reconstruction module 50. As described above, the DAS 26 samples and digitizes the projection data acquired by the plurality of detectors 20. Next, the image reconstruction module 50 performs high-speed image reconstruction on the basis of the aforementioned sampled and digitized projection data. In some embodiments, the image reconstruction module 50 stores the reconstructed image in a storage apparatus or a mass memory 46. Alternatively, the image reconstruction module 50 transmits the reconstructed image to a computer 40 to generate information for diagnosing and evaluating patients.

In some embodiments, the medical imaging system includes a scanning module, the scanning module including an X-ray source, a detector array, and an image reconstruction module. The scanning module can be used to transmit and receive X-rays at a scout scan stage to obtain scout images, and to control rotation of the rack and transmit and receive X-rays at a main scan stage to obtain medical images.

Although the image reconstruction module 50 is illustrated as a separate entity in FIG. 1, in some embodiments, the image reconstruction module 50 may form part of the computer 40. Alternatively, the image reconstruction module 50 may not exist in the system 10, or the computer 40 may perform one or more functions of the image reconstruction module 50. Furthermore, the image reconstruction module 50 may be located at a local or remote location and may be connected to the system 10 using a wired or wireless communication network. In some embodiments, centralized computing resources in a cloud communication network may be used in the image reconstruction module 50.

In some embodiments, the system 10 includes a control mechanism 30. The control mechanism 30 may include an X-ray controller 34 configured to provide power and timing signals to the X-ray radiation source 14. The control mechanism 30 may further include a rack controller 32 used to control the rotational speed and/or position of the rack 12 on the basis of imaging requirements. The control mechanism 30 may further include a carrier table controller 36 used to drive a carrier table 28 to move to a suitable position so as to position the subject under examination in the rack 12, in order to acquire projection data of the target volume of the subject under examination. Furthermore, the carrier table 28 includes a driving device, and the carrier table controller 36 may control the carrier table 28 by controlling the driving device.

In some embodiments, the system 10 further includes the computer 40, wherein data sampled and digitized by the DAS 26 and/or an image reconstructed by the image reconstruction module 50 is transmitted to a computer or the computer 40 for processing. In some embodiments, the computer 40 stores the data and/or image in a storage apparatus such as a mass memory 46. The mass memory 46 may include a hard disk drive, a floppy disk drive, a CD-read/write (CD-R/W) drive, a digital versatile disc (DVD) drive, a flash drive, and/or a solid-state storage apparatus. In some embodiments, the computer 40 may be connected to a local or remote display, printer, workstation and/or similar device, for example, connected to devices such as devices of medical institutions or hospitals, or connected to a remote device by means of one or more configured wires or a wireless communication network such as the Internet and/or a virtual private communication network.

In some embodiments, the computer 40 transmits the reconstructed image and/or other information to a display 42, the display 42 being communicatively connected to the computer 40 and/or the image reconstruction module 50. In some embodiments, the display 42 can include any form of display screen, wherein the display 42 can be a display screen that is located in a scan room, a main display screen that is located in a control room, or a mobile display. The display 42 includes a graphical user interface, and the graphical user interface can be used to display one or more among information about the subject under examination, the display and options of scanning protocols, scout images, and medical images.

Furthermore, the computer 40 may provide commands and parameters to the DAS 26 and the control mechanism 30 (including the rack controller 32, the X-ray controller 34, and the carrier table controller 36) on the basis of user provision and/or system definition, so as to control a system operation, such as data acquisition and/or processing. In some embodiments, the computer 40 controls system operation on the basis of user input. For example, the computer 40 may receive user input such as commands, scanning protocols and/or scanning parameters, by means of an operator console 48 connected thereto. The operator console 48 may include a keyboard (not shown) and/or touch screen to allow a user to input/select commands, scanning protocols and/or scanning parameters. Although FIG. 1 exemplarily shows only one operator console 48, the computer 40 may be connected to more operating consoles, for example, for inputting or outputting system parameters, requesting medical examinations, and/or viewing images.

In some embodiments, the operator console 48 may include a user interface module (or user input apparatus) having a certain form of operator interface, such as a keyboard, a mouse, a voice activated controller, or any other suitable input device, wherein an operator may input an operation signal/control signal to the computer by means of the user interface module. Specifically, the operator can perform selection and/or input of scanning protocols by means of the user interface module, and can perform selection and/or input of image noise parameters. Of course, the operator can also perform various operations such as editing and/or printing of medical images by means of the user interface module. In some embodiments, options such as scanning protocols and image noise parameters are displayed by means of a display, and the operator can perform corresponding operations by means of the user interface module.

In some embodiments, the system 10 may include or be connected to an image storage and transmission system (PACS) (not shown in the figure). In some embodiments, the PACS is further connected to a remote system such as a radiology information system, a hospital information system, and/or an internal or external communication network (not shown) to allow operators at different locations to provide commands and parameters and/or access image data.

The method or process described further below may be stored as executable instructions in a non-volatile memory in a computing device of the system 10. For example, the computer 40 may include the executable instructions in the non-volatile memory, and may use the method described herein to automatically perform part or all of the scanning process, for example, selecting suitable protocols and determining suitable parameters. As another example, the image reconstruction module 50 may include the executable instructions in the non-volatile memory, and may use the method described herein to perform image reconstruction tasks.

The computer 40 may be configured and/or arranged for use in different manners. For example, in some implementations, a single computer 40 may be used; in other implementations, a plurality of computers 40 are configured to work together (for example, on the basis of distributed processing configuration) or separately, wherein each computer 40 is configured to handle specific aspects and/or functions, and/or to process data for generating models used only for a specific medical imaging system 10. In some implementations, the computer 40 may be local (for example, in the same position as one or more medical imaging systems 10, for example, in the same facility and/or the same local communication network); in other implementations, the computer 40 may be remote and thus can only be accessed by means of a remote connection (for example, by means of the Internet or other available remote access technologies).

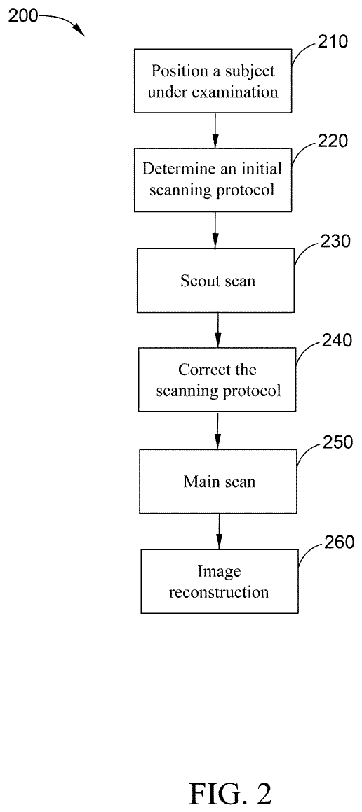

FIG. 2 shows a schematic diagram of a CT scanning process 200 according to some embodiments of the present invention. As shown in FIG. 2, in a complete CT scan, the following steps are generally required: positioning a subject under examination 210, determining an initial scanning protocol 220, performing a scout scan 230, correcting a scanning protocol 240, performing a main scan 250 and performing image reconstruction 260.

In some embodiments, it is first necessary to position a subject under examination 210. Specifically, it is necessary to make a selection in a list of patients, and then to obtain information about the subject under examination to obtain information such as age, image-capture site, etc., and then, according to the image-capture site etc., the subject under examination is placed in a suitable position, for example, in an anteroposterior position or lateral position, or, for example, lying in the middle of a bed, and so on. In some embodiments, auxiliary positioning may also be performed by a camera or a video camera disposed within a scan room, and information about the contour and/or thickness, etc., of the subject under examination may further be obtained on the basis of the camera or video camera.

Then, the scanning protocol is determined or obtained automatically or manually on the basis of the information about the subject under examination 220. Specifically, for example, a scanning protocol applicable to a child or an adult may be determined according to the age thereof, and different scanning protocols applicable to the brain or chest, etc., are determined according to, e.g., the image-capture site. Of course, the scanning protocol may also be manually or automatically confirmed according to other information or a combination of the described information, for example various pieces of information such as thickness, weight or size. The scanning protocol may be automatically confirmed by the computer according to the information about the subject under examination, or may be manually input or selected by the operator, or obtained by the operator according to the modification of a default or recommended scanning protocol.

In some embodiments, a plurality of scanning protocols are provided in the medical imaging system. The computer or controller can automatically recommend or display the optimal scanning protocol in the display on the basis of the information about the subject under examination. In some embodiments, a machine learning model is provided in the medical imaging system, which performs learning on the basis of sample data, so as to obtain, according to the information about the subject under examination, a corresponding scanning protocol.

The scanning protocol determined in step 220 includes a scanning protocol of the scout scan and a scanning protocol of the main scan. Specifically, the scanning parameters include at least one of a scan field of view (SFOV), a diagnostic purpose, a ray bowtie filter, a collimation width, an exposure voltage, a rack rotation speed, a slice thickness, and a helical pitch, and the image reconstruction parameters include at least one of a convolution kernel size for reconstruction, a display field of view (DFOV), and image post-processing parameters. The image post-processing parameters include the selection of algorithms for image denoising and related parameters, and the diagnostic purpose includes different scanning sites or scanning requirements, etc., such as chest scanning, cardiac scanning, hepatic angiography scanning, and the like. Specifically, in applications, the diagnostic purpose is quantified, e.g., using different numbers to represent different scanning sites or scanning requirements, so as to facilitate the training and application of the machine learning model.

Then, after the scanning protocol is confirmed, a scout scan is performed 230. In the scout scan, exposure may be performed with a lower dose at a fixed angle to obtain a scout image, a region of interest (ROI) can be automatically or manually determined according to the obtained scout image, and then, on the basis of the region of interest, the scanning position or coordinates are confirmed and the scanning protocol of the main scan is corrected or adjusted. Specifically, the scout scan can be performed at an angle of 0 degrees to obtain an anteroposterior (AP direction) image, wherein 0 degrees refers to the angle in which the X-ray source is located directly above. Alternatively, the scout scan can be performed at an angle of 90 degrees to obtain a lateral (LAT direction) image. Of course, the scan can be performed at both angles to obtain two scout images.

Secondly, after the scout image is obtained, the scanning protocol is corrected, and then on the basis of the corrected scanning protocol, the main scan is performed 250 and the image reconstruction is performed 260. In some embodiments, the data obtained by the main scan may be reconstructed on the basis of the selected image reconstruction parameters, and the computer may further edit, save, and/or print the medical images.

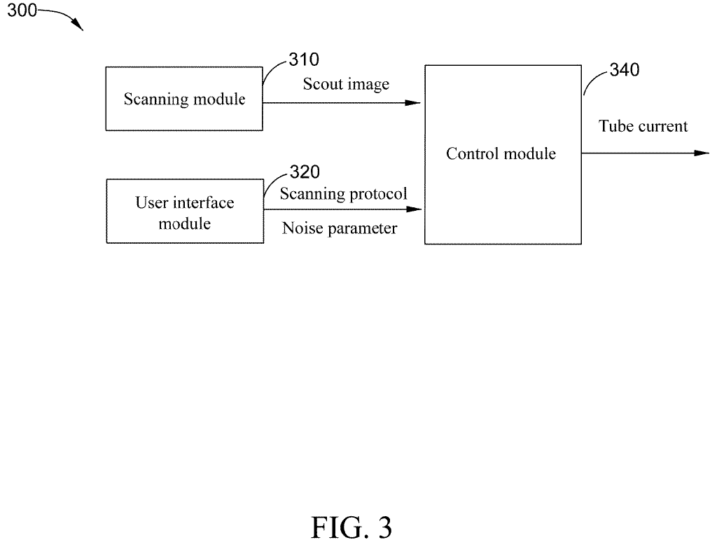

In some embodiments, the tube current value needs to be set or corrected in the correction process of the scanning protocol. In some embodiments, in order to further optimize or adjust a tube current value, FIG. 3 shows a schematic diagram of a medical imaging system 300 according to some embodiments of the present invention. As shown in FIG. 3, the medical imaging system 300 of the present invention includes a scanning module 310, a user interface module 320 and a control module 340. The scanning module 310 is configured to perform a scout scan to obtain a scout image, and to perform a main scan to obtain a medical image. The user interface module 320 is configured to select a scanning protocol and to select a preset image noise parameter. The control module 340 may be part of the computer shown in FIG. 1, or may be a cloud control module. The control module 340 is connected to the scanning module 310 and the user interface module 320, so as to obtain a tube current value on the basis of the trained machine learning model, according to the scout image, the scanning protocol, and the image noise parameter.

Specifically, the scout image is obtained from the scout scan process 230. The control module can further extract features from the scout image and input the extracted features into the machine learning model. In some embodiments, feature extraction may be achieved by performing image segmentation or image identification on the basis of the machine learning model, or may be achieved by performing image processing the scout image. In some embodiments, the features of the scout image may be data obtained on the basis of processing or operating the scout image itself, or data obtained by processing or extracting information stored in a corresponding header file of the scout image.

Specifically, the extracted features include at least one among total attenuation, peak attenuation, water-equivalent diameter, elliptical ratio, width of human body contour, proportion of low attenuation tissue, proportion of medium attenuation tissue, and proportion of high attenuation tissue in the scout images. In some embodiments, any of the above features includes a group of feature data sets, each group of data sets being determined according to the scan direction. For example, for a two-dimensional scout image, the width of a human body contour in each row or each column on the image is calculated separately, and the width of the human body contour includes a combination or a collection of widths of the human body contour in all rows or all columns.

The scanning protocol is confirmed in the process 220, and the options of the scanning protocol may be displayed in the display and related modifications or confirmations are made on the basis of the input of the user (by means of the user interface module).

Related selection or confirmation of the image noise parameter is also made on the basis of the input of the user (by means of the user interface module). In some embodiments, the image noise parameter may be confirmed in the process of confirming the scanning protocol, or may be confirmed after performing the scout scan, and there are no specific requirements for the order of selection of the image noise parameter. The scanning parameters include at least one among scan field of view, diagnostic purpose, ray bowtie filter, collimation width, exposure voltage, rack rotation speed, slice thickness and helical pitch, and the image reconstruction parameters include at least one among convolution kernel size for reconstruction, display field of view, and image post-processing parameters.

In some embodiments, the noise parameter used in the present application is a global noise index (GNI). The global noise index is an indicator of image noise, and is determined by means of an image histogram peak. In particular, a histogram is obtained by calculating the variance for all pixel values in a medical image and performing statistical calculation. The peak of the histogram is representative of most of the noise of the medical image, that is, the global noise index. By selecting a desired noise level, the noise level of the ultimately obtained medical image can be confirmed, and the machine learning model can accordingly output a corresponding tube current value.

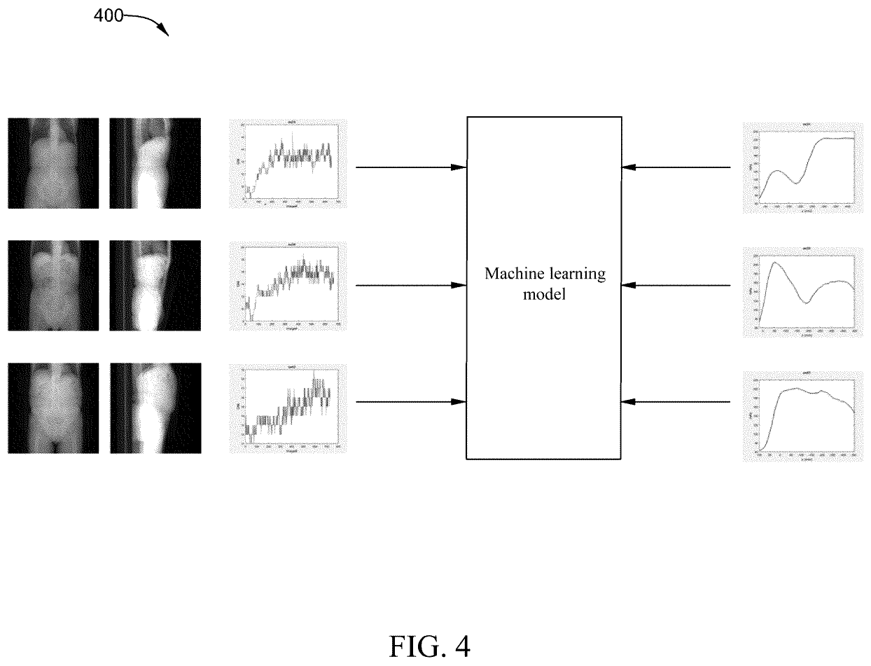

FIG. 4 shows a schematic diagram of training of the machine learning model in the control module shown in FIG. 3. As shown in FIG. 4, for ease of illustration, the features or parameters of the scanning protocol are omitted in FIG. 4. In some embodiments, the machine learning model of the present application includes a linear regression model that is obtained by training using a clinical data set. The training data set includes clinical data of a plurality of subjects under examination. The clinical data of each subject under examination includes a scout image, a scanning protocol, an image noise parameter of a medical image, and a tube current value of actual scanning.

The machine learning model of the present application is obtained by training using actual clinical data. In clinical practice, scout images, scanning protocols, and medical images are all required, and relevant data and information are stored; for example, these may be stored in a medical imaging system, or may be stored in a picture archiving and communication system (PACS), or stored in a cloud. The use of clinical data can effectively overcome problems caused by an equivalent phantom.

Specifically, the training includes: obtaining a clinical scout image, a scanning protocol, an image noise parameter, and a tube current value of actual scanning of each subject under examination; and performing training using the clinical scout images, scanning protocols and image noise parameters of medical images as an input and using tube current values as an output, so as to obtain the machine learning model.

The scout image and medical image are analyzed; for example, the actual image noise parameter of the medical image is calculated, and then a tube current value used in the current scanning is extracted from the actually used scanning protocol. The scanning protocol, features in the scout image, image noise parameter, and tube current value of actual scanning corresponding to each clinical scanning are input into a machine learning model, wherein the scanning protocol, features in the scout image, and image noise parameter are used as a preset input, i.e., x, and the tube current value of actual scanning is used as a preset output, i.e., y. By means of machine learning, a functional relationship of the tube current value y with the scanning protocol and features in the scout image and image noise parameter x can be obtained, that is, y=f (x). In application, by confirming the machine learning model, as long as a scanning protocol, a scout image, and a preset noise parameter are input into the machine learning model, a corresponding tube current value can be obtained. In some embodiments, the features extracted from at least one image among the AP scout image and the LAT scout image may be input into the machine learning model.

In some embodiments, the linear regression model in the present application can be confirmed by using the equation of some embodiments as follows: specifically, the tube current value

$$mA ( z ) = exp ( {[ \begin{matrix}{a}_{0} & {a}_{1} & {a}_{2} & … & {a}_{n}\end{matrix} ] [ \begin{matrix}{TA}_{AP} ( z ) , {PA}_{AP} ( z ) , {WED}_{AP} ( z ) , {OR}_{AP} ( z ) , {Width}_{AP} ( z ) , {R}_{{Low}_{AP}} ( z ) , {R}_{{Med}_{AP}} ( z ) , {R}_{{High}_{AP}} ( z ) , \\ {TA}_{Lat} ( z ) , {PA}_{Lat} ( z ) , {WED}_{Lat} ( z ) , {OR}_{Lat} ( z ) , {Width}_{Lat} ( z ) , {R}_{{Low}_{Lat}} ( z ) , {R}_{{Med}_{Lat}} ( z ) , {R}_{{High}_{Lat}} ( z ) , \\ k V , Bowtie , Helical Pitch , Slice Thickness , Recon Kernel , ln ( GNI )\end{matrix} ]}^{T} )$$

Wherein [a₀a₁a₂ . . . aₙ] represents the functional relationship obtained by training f, Z represents the scanning direction, TA_{AP(z)} represents the total attenuation in the AP scout image, PA_{AP(z)} represents the peak attenuation in the AP scout image, WED_{AP(z)} represents the water-equivalent diameter in the AP scout image, OR_{AP(z)} represents the elliptical ratio in the AP scout image, Width_{AP(z)} represents the width of human body contour in the AP scout image, R_{Low}_{_{AP(z)}} represents the proportion of low attenuation tissue in the AP scout image, R_{Med}_{_{AP}}(z) represents the proportion of medium attenuation tissue in the AP scout image, R_{High}_{_{AP(z)}} represents the proportion of high attenuation tissue in the AP scout image, TA_{lat(z)} represents the total attenuation in the LAT scout image, PA_{lat(z)} represents the peak attenuation in the LAT scout image, WED_{lat(z)} represents the water-equivalent diameter in the LAT scout image, OR_{lat(z)} represents the elliptical ratio in the LAT scout image, Width_{lat(z)} represents the width of human body contour in the LAT scout image, R_{Low} _{_{lat(z)}} represents the proportion of low attenuation tissue in the LAT scout image, R_{Med}_{ₗₐₜ} (z) represents the proportion of medium attenuation tissue in the LAT scout image, R_{High}_{_{lat(z)}} represents the proportion of high attenuation tissue in the LAT scout image, kV represents the exposure voltage, bowtie represents the ray bowtie filter, Helical Pitch represents the helical pitch, Slice Thickness represents the slice thickness, Recon Kernel represents the convolution kernel size for image reconstruction, and In(GNI) represents the inputted global noise index parameter.

In some embodiments, the machine learning model may not only include a linear regression model, but may also include any other suitable machine learning model, e.g., deep learning. In some embodiments, the features of the scout image, as well as the scanning parameter and image reconstruction parameter used in the linear regression model are not limited to the selections described above, and any other suitable parameters or features may also be used; for example, a diagnostic purpose parameter and slice thickness parameter can be added, and a helical pitch parameter may not be used, and the like.

In some embodiments, the calculation of the image noise parameter may be a calculation of a global noise GNI of the medical image, to obtain an actual global noise value of the medical image. Of course, the medical image may also be segmented on the basis of (on the basis of artificial intelligence or machine learning) to obtain an image of a relevant site or region of interest, and the noise value corresponding to the site or ROI is calculated. And of course, the medical image may be segmented or processed by an experienced operator to obtain the image noise parameter of the segmented region. It should be understood by a person skilled in the art that the image noise parameter mentioned in the present application is not limited to the several solutions mentioned above, and any other suitable computation method may also be used, as long as the noise corresponding to the medical image obtained by the actual scanning is satisfied.

In some embodiments, the features of the scout image may also include at least one among a plurality of features extracted from the scout image and multiple pieces of information identified or extracted from the header file, and the number of relevant input x is not limited, so long as the input in actual application corresponds to the input x used in training or learning.

In some embodiments, the machine learning model can be further optimized. For example, a set of data that was scanned using the machine learning model to obtain the tube current value may be input again into the machine learning model, so as to further optimize the machine learning model. Of course, the machine learning model can also self-learn by utilizing transfer learning, to further improve the performance of the machine learning model.

In some embodiments, once the training of the machine learning model is completed, the machine learning model is copied and/or loaded into the medical imaging system, which may be completed in different ways. For example, models may be loaded by means of a directional connection or link between the medical imaging system 10 and the computer 40. In this regard, communication between different elements may be accomplished using an available wired and/or wireless connection and/or according to any suitable communication (and/or network) standard or protocol. Alternatively or additionally, the data may be indirectly loaded into the medical imaging system 10. For example, the data may be stored in a suitable machine-readable medium (for example, a flash memory card), and then the medium is used to load the data into the medical imaging system 10 (for example, by a user or an authorized personnel of the system on site); or the data may be downloaded to an electronic apparatus (for example, a laptop) capable of local communication, and then the apparatus is used on site (for example, by a user or an authorized personnel of the system) to upload the data to the medical imaging system 10 by means of a direct connection (for example, a USB connector).

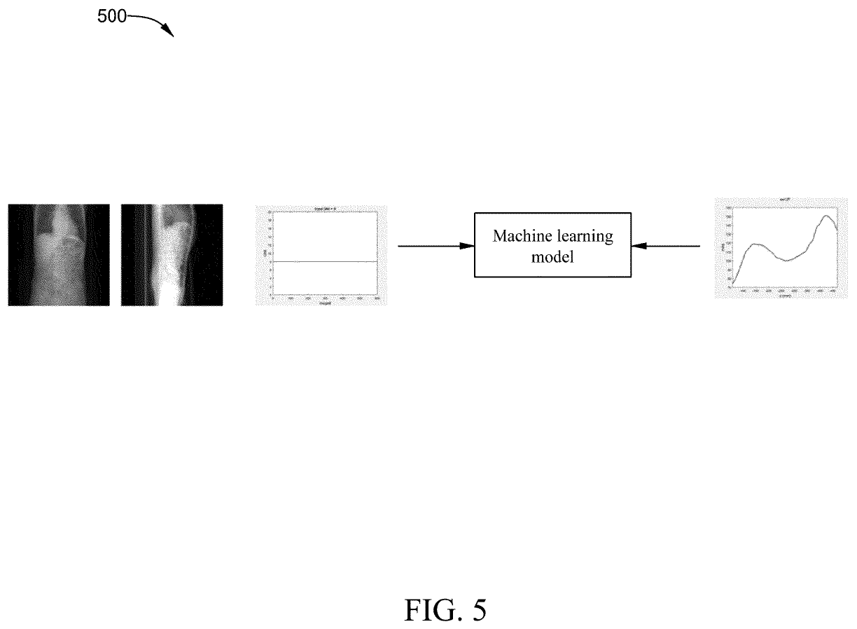

FIG. 5 shows a schematic diagram of an application of the machine learning model in the control module shown in FIG. 3. As shown in FIG. 5, for ease of description and illustration, the parameters of the scanning protocol are omitted in FIG. 5. The control module may input a scanning protocol, a scout image, and a preset noise parameter into a machine learning model, wherein a corresponding tube current value can be outputted or obtained, and the scanning protocol is corrected on the basis of the tube current value, and then a main scan is performed on the basis of the corrected scanning protocol. The preset noise parameter is inputted into the control module by the user by means of a user operation module.

Specifically, carrying out training and learning using known clinical data can resolve errors between the actual human body and an equivalent phantom, and the analysis of a tube current value of actual scanning also solves the problem of large errors in relevant tests using a human phantom in the laboratory.

In some embodiments, a display in the medical imaging system in the present application includes a graphical user interface which includes a plurality of setting interfaces. The plurality of setting interfaces includes a selection or adjustment interface for displaying scanning protocols, and a selection or adjustment interface for image noise parameters. Of course, the image user interface can also display scout images and/or medical images obtained from a main scan. In some embodiments, the display may be a touch screen. The operator may perform related control or operations by touch, or by an external user interface module.

In some embodiments, the medical imaging system of some embodiments of the present application includes a processor. The processor can obtain a scanning protocol, control a scanning component of the medical imaging system to perform a scout scan so as to obtain a scout image, extract features from the scout image to obtain the features of the scout image, and then obtain a tube current value on the basis of the trained machine learning model, according to the features of the scout image, the scanning protocol, and a preset image noise parameter. Furthermore, the processor can also, on the basis of the obtained tube current value, control the scanning component to perform a main scan, so as to obtain a medical image of the subject under examination.

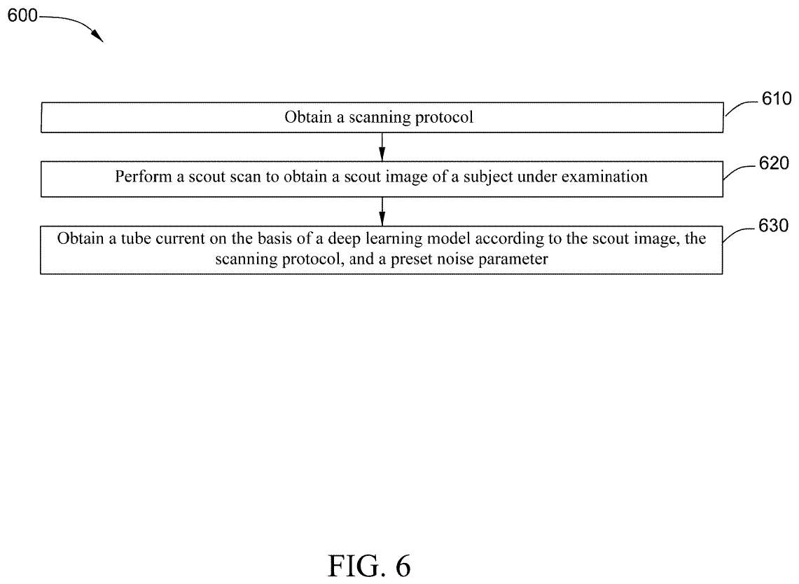

FIG. 6 shows a flowchart of a method 600 for obtaining a tube current value according to some embodiments of the present invention. As shown in FIG. 6, the method 600 for obtaining a tube current value includes step 610, step 620, and step 630.

In step 610, a scanning protocol is obtained. In some embodiments, the scanning protocol includes a scanning parameter and an image reconstruction parameter. The scanning parameter includes at least one among a scan field of view, a diagnostic purpose, a ray bowtie filter, a collimation width, an exposure voltage, a rack rotation speed, a slice thickness and a helical pitch, and the image reconstruction parameter includes at least one among a convolution kernel size for reconstruction, a display field of view, and an image post-processing parameter.

Specifically, the scanning protocol can be determined or obtained automatically or manually on the basis of information about a subject under examination. In some embodiments, a plurality of scanning protocols are provided in the medical imaging system. The computer or controller can automatically recommend or display the optimal scanning protocol in the display on the basis of the information about the subject under examination. In some embodiments, a machine learning model is provided in the medical imaging system, which performs learning on the basis of sample data so as to obtain a corresponding scanning protocol according to the information about the subject under examination. In step 620, a scout scan is performed to obtain a scout image of the subject under examination.

Specifically, the scout image includes at least one among an AP image and a LAT image. Specifically, the scout scan can be performed at an angle of 0 degrees to obtain an anteroposterior image, wherein 0 degrees refers to the angle in which the X-ray source is located directly above. Alternatively, the scout scan can be performed at an angle of 90 degrees to obtain a lateral image. Of course, the scan can be performed at multiple angles to obtain multiple scout images.

In some embodiments, the method for obtaining a tube current value further includes extracting features from the scout image. In some embodiments, the feature extraction may be achieved by performing image segmentation or image identification on the basis of the machine learning model, or may be achieved by performing image processing on the scout image. In some embodiments, the features of the scout image may be data obtained on the basis of processing or operation of the scout image itself, or data obtained by processing or extracting information stored in a corresponding header file of the scout image. The extracted features include at least one among total attenuation, peak attenuation, water-equivalent diameter, elliptical ratio, width of human body contour, proportion of low attenuation tissue, proportion of medium attenuation tissue, and proportion of high attenuation tissue in the scout images.

In some embodiments, the method for obtaining a tube current value further includes obtaining an image noise parameter. Specifically, the preset image noise parameter includes a global image noise parameter. Specifically, related selections or confirmations of the image noise parameter are also made on the basis of the input of the user (by means of the user interface module).

In step 630, the tube current value is obtained on the basis of the trained machine learning model according to the scout image, the scanning protocol and the preset image noise parameter. Specifically, the machine learning model includes a linear regression model. Specifically, the machine learning model is obtained by training using a clinical data set. The training data set includes clinical data of a plurality of subjects under examination. The clinical data of each subject under examination includes a scout image, a scanning protocol, an image noise parameter of a medical image, and a tube current value of actual scanning.

Specifically, the training includes: obtaining a clinical scout image, scanning protocol, image noise parameter, and tube current value of actual scanning of each subject under examination; and performing training using the clinical scout images, scanning protocols and image noise parameters of medical images as an input and using the tube current values as an output, so as to obtain the machine learning model.

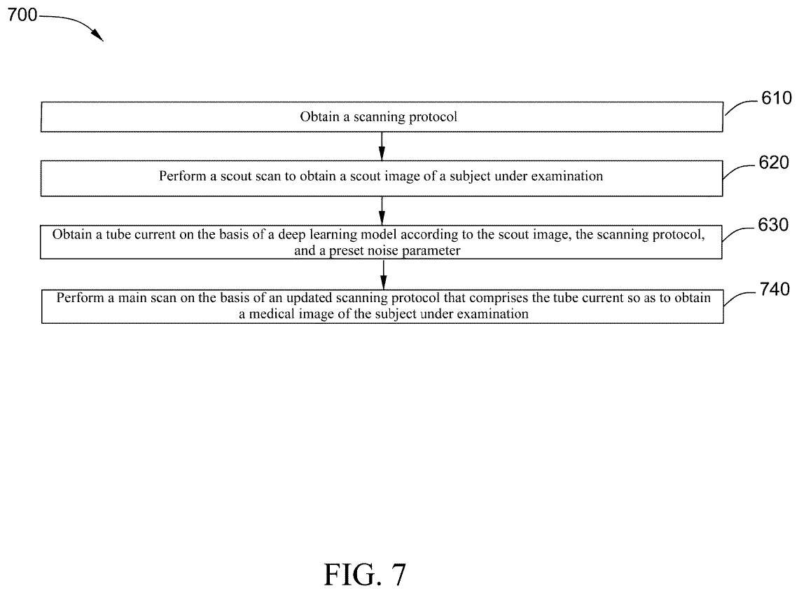

FIG. 7 shows a flowchart of a CT scanning method 700 according to some embodiments of the present invention. As shown in FIG. 7, as compared to the method 600 for obtaining a tube current value as shown in FIG. 6, the CT scanning method shown in FIG. 7 further includes step 740.

In step 740, a CT scan is performed on the basis of an updated scanning protocol corresponding to the tube current value, to obtain a medical image of the subject under examination. According to the method for obtaining a tube current value proposed in the present invention, first, a tube current value is corrected or adjusted using a machine learning model, so that the problem of large errors when performing correction using an equivalent phantom is avoided and the process is simplified. Secondly, in the process of training the machine learning model, previously saved or stored clinical data is used, so that the machine learning model can be more accurate, and more suitable for the complex structure of the human body, and the quality of the resulting medical images is higher.

The present invention may further provide a non-transitory computer-readable storage medium, which is used for storing an instruction set and/or a computer program. When executed by a computer, the instruction set and/or computer program causes the computer to perform the aforementioned method for obtaining a tube current value. The computer executing the instruction set and/or computer program may be a computer of a medical imaging system, or may be other apparatuses/modules of the medical imaging system. In one embodiment, the instruction set and/or computer program may be programmed into a processor/controller of the computer.

Specifically, when executed by the computer, the instruction set and/or computer program causes the computer to: obtain a scanning protocol; perform a scout scan to obtain a scout image of a subject under examination; and obtain the tube current value on the basis of a trained machine learning model according to the scout image, the scanning protocol, and a preset image noise parameter.

The instructions described above may be combined into one instruction for execution, and any of the instructions may also be split into a plurality of instructions for execution. Moreover, the present invention is not limited to the instruction execution order described above.

An exemplary embodiment of the present invention provides a method for obtaining a tube current value, the method comprising: obtaining a scanning protocol; performing a scout scan to obtain a scout image of a subject under examination; and obtaining a tube current value on the basis of a trained machine learning model according to the scout image, the scanning protocol, and a preset image noise parameter.

Specifically, the scanning protocol includes a scanning parameter and an image reconstruction parameter. The scanning parameter includes at least one among a scan field of view, a diagnostic purpose, a ray bowtie filter, a collimation width, an exposure voltage, a rack rotation speed, a slice thickness and a helical pitch, and the image reconstruction parameter includes at least one among a convolution kernel size for reconstruction, a display field of view, and an image post-processing parameter. Specifically, the preset image noise parameter includes a global image noise parameter. Specifically, the machine learning model includes a linear regression model.

Specifically, the machine learning model is trained using a clinical data set. The clinical data set includes clinical data of a plurality of subjects under examination. Each clinical data includes a scout image, a scanning protocol, an image noise parameter of a medical image, and a tube current value of actual scanning.

Specifically, the training includes obtaining a clinical scout image, scanning protocol, image noise parameter, and tube current value of actual scanning of each subject under examination, and performing training using the clinical scout images, scanning protocols and image noise parameters of medical images as an input and using the tube current values as an output, so as to obtain the machine learning model.

Specifically, the method further includes extracting features from the scout images, and inputting the extracted features into the machine learning model to obtain the tube current value. Specifically, the extracted features include at least one among total attenuation, peak attenuation, water-equivalent diameter, elliptical ratio, width of human body contour, proportion of low attenuation tissue, proportion of medium attenuation tissue, and proportion of high attenuation tissue in the scout images.

An exemplary embodiment of the present invention further provides a CT scanning method, the method comprising: obtaining a scanning protocol; performing a scout scan to obtain a scout image of a subject under examination; obtaining a tube current value on the basis of a trained machine learning model according to the scout image, the scanning protocol, and a preset image noise parameter, so as to obtain an updated scanning protocol; and performing a CT scan on the basis of the updated scanning protocol to obtain a medical image of the subject under examination.

Specifically, the scanning protocol includes a scanning parameter and an image reconstruction parameter. The scanning parameter includes at least one among a scan field of view, a diagnostic purpose, a ray bowtie filter, a collimation width, an exposure voltage, a rack rotation speed, a slice thickness and a helical pitch, and the image reconstruction parameter includes at least one among a convolution kernel size for reconstruction, a display field of view, and an image post-processing parameter.

Specifically, the preset image noise parameter includes a global image noise parameter. Specifically, the machine learning model includes a linear regression model. Specifically, the machine learning model is trained using a clinical data set. The clinical data set includes clinical data of a plurality of subjects under examination. Each clinical data includes a scout image, a scanning protocol, an image noise parameter of a medical image, and a tube current value of actual scanning.

Specifically, the training includes obtaining a clinical scout image, scanning protocol, image noise parameter, and tube current value of actual scanning of each subject under examination, and performing training using the clinical scout images, scanning protocols and image noise parameters of medical images as an input and using the tube current values as an output, so as to obtain the machine learning model.

Specifically, the method further includes extracting features from the scout images, and inputting the extracted features into the machine learning model to obtain the tube current value. Specifically, the extracted features include at least one of total attenuation, peak attenuation, water-equivalent diameter, elliptical ratio, width of human body contour, proportion of low attenuation tissue, proportion of medium attenuation tissue, and proportion of high attenuation tissue in the scout images.

An exemplary embodiment of the present invention further provides a medical imaging system, the system including a processor, the processor obtaining a scanning protocol, performing a scout scan to obtain a scout image of a subject under examination, and obtaining a tube current value on the basis of a trained machine learning model according to the scout image, the scanning protocol, and a preset image noise parameter.

Specifically, the scanning protocol includes a scanning parameter and an image reconstruction parameter. The scanning parameter includes at least one among a scan field of view, a diagnostic purpose, a ray bowtie filter, a collimation width, an exposure voltage, a rack rotation speed, a slice thickness and a helical pitch, and the image reconstruction parameter includes at least one among a convolution kernel size for reconstruction, a display field of view, and an image post-processing parameter.

Specifically, the preset image noise parameter includes a global image noise parameter. Specifically, the machine learning model includes a linear regression model. Specifically, the machine learning model is trained using a clinical data set. The clinical data set includes clinical data of a plurality of subjects under examination. Each clinical data includes a scout image, a scanning protocol, an image noise parameter of a medical image, and a tube current value of actual scanning.

Specifically, the training includes obtaining a clinical scout image, scanning protocol, image noise parameter, and tube current value of actual scanning of each subject under examination, and performing training using the clinical scout images, scanning protocols and image noise parameters of medical images as an input and using the tube current values as an output, so as to obtain the machine learning model.

Specifically, the processor is further configured to extract features from the scout images, and to input the extracted features into the machine learning model to obtain the tube current value. Specifically, the extracted features include at least one among total attenuation, peak attenuation, water-equivalent diameter, elliptical ratio, width of human body contour, proportion of low attenuation tissue, proportion of medium attenuation tissue, and proportion of high attenuation tissue in the scout images.

An exemplary embodiment of the present invention further provides a medical imaging system, the medical imaging system comprising: a scanning module, a user interface module and a control module; the scanning module is configured to perform a scout scan to obtain a scout image, and to perform a main scan to obtain a medical image; a user interface module is configured to select a scanning protocol and to select a preset image noise parameter; and a control module is configured to obtain a tube current value on the basis of a trained machine learning model, according to the scout image, the scanning protocol, and the image noise parameter.

Specifically, the scanning protocol includes a scanning parameter and an image reconstruction parameter. The scanning parameter includes at least one among a scan field of view, a diagnostic purpose, a ray bowtie filter, a collimation width, an exposure voltage, a rack rotation speed, a slice thickness and a helical pitch, and the image reconstruction parameter includes at least one among a convolution kernel size for reconstruction, a display field of view, and an image post-processing parameter.

Specifically, the preset image noise parameter includes a global image noise parameter. Specifically, the machine learning model includes a linear regression model. Specifically, the machine learning model is trained using a clinical data set. The clinical data set includes clinical data of a plurality of subjects under examination. Each clinical data includes a scout image, a scanning protocol, an image noise parameter of a medical image, and a tube current value of actual scanning.

Specifically, the training includes obtaining a clinical scout image, scanning protocol, image noise parameter, and tube current value of actual scanning of each subject under examination, and performing training using the clinical scout images, scanning protocols and image noise parameters of medical images as an input and using the tube current values as an output, so as to obtain the machine learning model.

Specifically, the control module is further configured to extract features from the scout images, and to input the extracted features into the machine learning model to obtain the tube current value. Specifically, the extracted features include at least one among total attenuation, peak attenuation, water-equivalent diameter, elliptical ratio, width of human body contour, proportion of low attenuation tissue, proportion of medium attenuation tissue, and proportion of high attenuation tissue in the scout images.

As used herein, the term “computer” may include any processor-based or microprocessor-based system, which includes a system that uses a microcontroller, a reduced instruction set computer (RISC), an application-specific integrated circuit (ASIC), a logic circuit, and any other circuit or processor capable of performing the functions described herein. The examples above are exemplary only and are not intended to limit the definition and/or meaning of the term “computer” in any way.

The instruction set may include various commands used to instruct the computer serving as a processing machine or the processor to perform specific operations, for example, methods and processes of various embodiments. The instruction set may be in the form of a software program that may form part of one or more tangible, non-transitory computer readable media. The software may be in various forms of, for example, system software or application software. Furthermore, the software may be in the form of a standalone program or a collection of modules, a program module within a larger program, or part of a program module. The software may also include modular programming in the form of object-oriented programming. Processing of input data by the processing machine may be in response to an operator command, or in response to a previous processing result, or in response to a request made by another processing machine.

Some exemplary embodiments have been described above; however, it should be understood that various modifications may be made. For example, suitable results can be achieved if the described techniques are performed in a different order and/or if components in the described systems, architectures, devices, or circuits are combined in different ways and/or replaced or supplemented by additional components or equivalents thereof. Accordingly, other implementations also fall within the scope of protection of the claims.

1. A method for obtaining a tube current value, comprising:
obtaining a scanning protocol;
performing a scout scan to obtain a scout image of a subject under examination; and
obtaining a tube current value on the basis of a trained machine learning model, according to the scout image, the scanning protocol, and a preset image noise parameter.

2. The method of claim 1, wherein the scanning protocol comprises a scan parameter and an image reconstruction parameter, the scan parameter comprising at least one of a scan field of view, a diagnostic purpose, a ray bowtie filter, a collimation width, an exposure voltage, a rack rotation speed, a slice thickness and a helical pitch, and the image reconstruction parameter comprising at least one of a convolution kernel size for reconstruction, a display field of view, and an image post-processing parameter.

3. The method of claim 1, wherein the preset image noise parameter comprises a global image noise parameter.

4. The method of claim 1, wherein the machine learning model comprises a linear regression model.

5. The method of claim 1, wherein the machine learning model is trained by means of a clinical data set, the clinical data set comprising clinical data of a plurality of subjects under examination, and each piece of clinical data comprising a scout image, a scanning protocol, an image noise parameter of a medical image, and a tube current value of actual scanning.

6. The method of claim 5, wherein the training comprises:
obtaining a clinical scout image, a scanning protocol, an image noise parameter, and a tube current value of actual scanning of each subject under examination; and
performing training using the clinical scout images, scanning protocols and image noise parameters of medical images as an input and using the tube current values as an output, so as to obtain the machine learning model.

7. The method of claim 1, further comprising:
extracting a feature from the scout image, and inputting the extracted feature into the machine learning model to obtain the tube current value.

8. The method of claim 7, wherein the extracted features comprise at least one of a total attenuation, a peak attenuation, a water-equivalent diameter, an elliptical ratio, a width of a human body contour, a proportion of low attenuation tissue, a proportion of medium attenuation tissue, and a proportion of high attenuation tissue in the scout image.

9. A CT scanning method, comprising:
determining a scanning protocol;
performing a scout scan to obtain a scout image of a subject under examination;
obtaining a tube current value on the basis of a trained machine learning model, according to the scout image, the scanning protocol, and a preset image noise parameter, so as to obtain an updated scanning protocol; and
performing a CT scan on the basis of the updated scanning protocol, to obtain a medical image of the subject under examination.

10. A medical imaging system, comprising a processor, the processor being configured to perform the method for obtaining a tube current value of any one of claim 1.

11. A medical imaging system, comprising:
a scanning module configured to perform a scout scan to obtain a scout image, and to perform a main scan to obtain a medical image;
a user interface module configured to select a scanning protocol and to select a preset image noise parameter; and
a control module configured to obtain a tube current value on the basis of a trained machine learning model, according to the scout image, the scanning protocol, and the image noise parameter.
